(19) United States
(12) Patent Application Publication
(10) Pub. No.: US 2007/0032776 A1
(43) Pub. Date: Feb. 8, 2007

(54) Extended locking luer connector

(76) Inventors: Allen W. Skinner, Florissant, MO (US); Toh Seng Goh, Ballwin, MO (US)

Correspondence Address: Bausch & Lomb Incorporated, One Bausch & Lomb Place, Rochester, NY, 14604-2701, US

(21) Appl. No.: 11/186,458

(22) Filed: Jul. 21, 2005

(51) Int. Cl. A61M 25/00

(52) U.S. Cl. 604/523

(57) ABSTRACT

A length of tubing 22 has a luer 25 connected to one end. The luer 25 includes a lumen 30 of a length to frictionally engage with and seat against a mating recess of a first ophthalmic surgical instrument. A locking hub 36, lockingly engages with a mating structure of a second surgical instrument. This structure then allows the length of tubing 22 to be alternately connected to the first and second ophthalmic surgical instruments and allows the length of tubing 22 to be used on multiple instruments during a single surgical procedure.

### BACKGROUND OF THE INVENTION

1. Field of the Invention

The present invention relates to mechanisms for connecting surgical tubing to a surgical handpiece. More specifically, the present invention is directed to a connector for connecting an infusion tube to a surgical handpiece.

2. Description of Related Art

There are two common means for connecting irrigation lines or tubes to a surgical handpiece. The first common method is a frictional fit shown in FIG. 1. A surgical handpiece 10, such as an irrigation/aspiration handpiece used in ophthalmic eye surgery is shown. An aspiration tube 12 is connected in a known manner to the handpiece. Irrigation tube 14 is connected to handpiece 10 via friction fit with handpiece generally at 16. Not shown is that a length of tube 14 extends into handpiece 10 to frictionally mate with a recess in handpiece 10.

The other common connector for connecting an irrigation line to a surgical handpiece is shown in FIG. 2. The connector of FIG. 2 shows an irrigation tube 18 having a locking luer 20 connected to the end of tube 18. Locking luer 20 typically has a short extension 22 which is typically of a length of about 0.09 inches and shown generally at arrow 24. Locking luer 20 is then twisted or threaded onto a mating connector structure of a surgical handpiece (not shown).

Both of the connection means shown in FIGS. 1 and 2 of the prior art provide for stable and effective connection to hand held surgical instruments, particularly for ophthalmic surgery. However, during surgery and, in particular ophthalmic surgery, it is often necessary to switch between various surgical handpieces, which each require an irrigation tube to be connected to the handpiece. Because one handpiece may require the frictional fit, such as shown in FIG. 1 and another handpiece may require the locking luer connector of FIG. 2, it is potentially necessary for a surgeon to cobble together an adapter to allow the use of both instruments; or the surgeon may simply be forced into using handpieces with the same irrigation connection means. In the field of ophthalmic surgery it is generally the industry standard to use standard frictional fit luer connections for irrigation tubes, however it is also known to use a standard locking luer on some. The luer standard is defined by ISO 594 parts 1 and 2. The choice between using an adapter, which may delay surgery, and being forced to use surgical handpieces that are not the surgeon's first preference is unappealing.

Heretofore, the extension 22 locking luer 20, as shown in FIG. 2, has an insufficient length 24 to allow for a friction fit in a handpiece 10, such as shown in FIG. 1. Handpiece 10 and similar instruments require a length greater than that found on known locking luer tube sets. If the extension 22 of a locking luer is of insufficient length, the extension 22 will not seat and seal within the mating recess of handpiece 10.

Therefore, it would be advantageous to have a single tube set that could be connected to the two types of surgical handpieces so that an irrigation connection could be quickly made from one instrument to the other during ophthalmic surgery. This would increase the efficiency of the surgery.

### BRIEF DESCRIPTION OF THE DRAWING

FIG. 1 is a prior art elevation showing a frictional irrigation tubing connection;

FIG. 2 is a partial perspective view showing a prior art locking luer connector;

FIG. 3 is a partial perspective view of a length of tubing in accordance with the present invention being connected to a surgical handpiece;

FIG. 4 is a partial elevation view of a length of tubing in accordance with the present invention and attached to a surgical handpiece; and

FIG. 5 is a partial perspective view of a length of tubing in accordance with the present invention for connection to a surgical handpiece requiring the use of the locking luer.

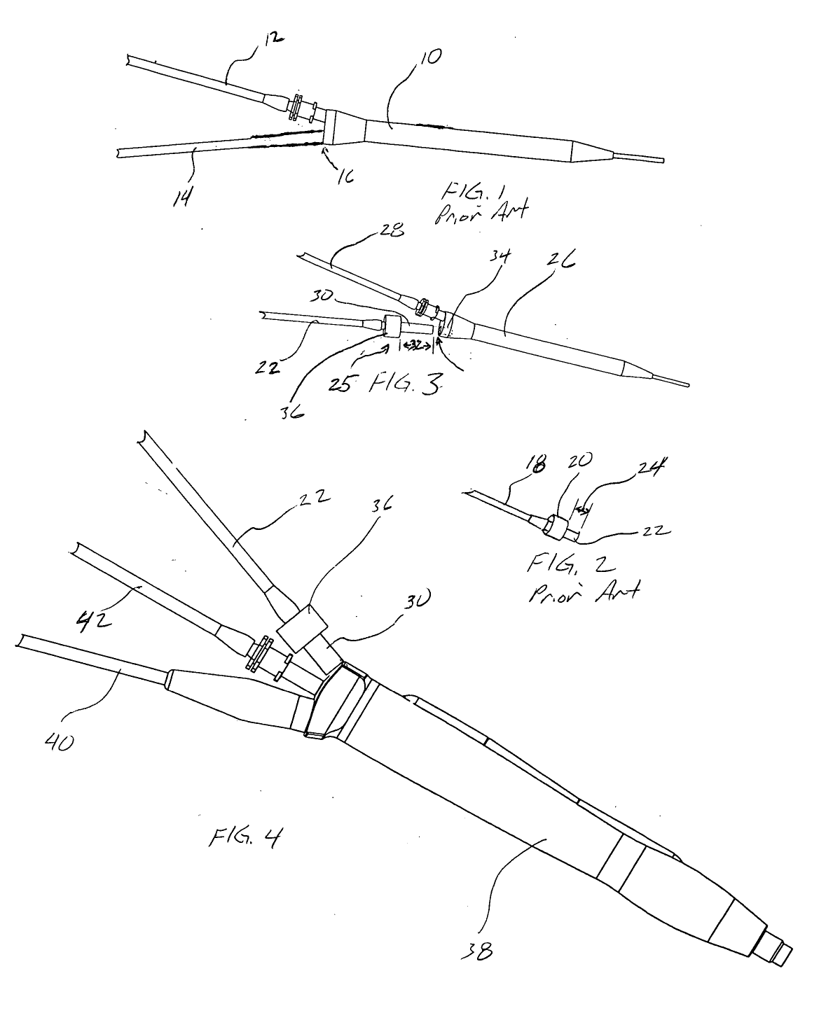

### DETAILED DESCRIPTION OF THE PREFERRED EMBODIMENT

FIG. 3 shows a length of tubing 22 having a luer 25 connected to an end of a length of tubing 22; while the other end of tubing 22 not shown, is typically connected to a surgical system, such as Bausch & Lomb Incorporated's Millennium Surgical System and specifically to a BSS bottle (also not shown). The end of tubing 22 with luer 25 is typically connected to a handpiece 26, which also includes aspiration tubing 28. Surgical handpiece 26 may be any of a variety of surgical handpieces, such as a phacoemulsification device, irrigation/aspiration device, or other surgical instruments requiring an irrigation connection.

Luer 25 includes a lumen 30 of sufficient length 32 to frictionally engage with and seat against a mating recess 34 of a first surgical instrument 26. Luer 25 also includes a locking hub 36 for locking engagement with a mating structure of a second surgical instrument described below, with reference to FIG. 5. Thus, luer 25 allows the length of tubing 22 to be alternately connected to the first and second surgical instruments and allows the length of tubing 22 to be used on multiple instruments during a single surgical procedure.

The lumen length 32 should typically be at least 0.47 inches in order to sealingly seat with the mating recess 34. In addition, lumen 30 is typically formed of plastic and has a slight taper that conforms to ISO 594 standard. Locking hub 36 preferably includes at least one thread for engaging a flange of the second ophthalmic instrument. Although a threaded connection onto a single flange has been shown other connections for locking luer 25 can be envisioned. By way of example, such connections may be snap-on connectors, detent connections, or any other connection means between the hub 36 and a mating structure on a surgical instrument.

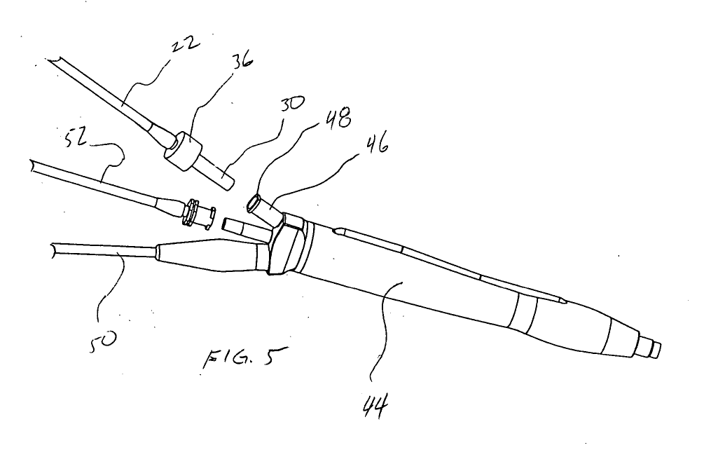

FIG. 4 shows tubing 22 with hub 36 and lumen 30 frictionally connected to a surgical handpiece 38; also shown are a power cord 40 and aspiration tubing 42.

FIG. 5 shows tubing 22 with hub 36 and lumen 30 in an exploded perspective view for locking engagement with a surgical handpiece 44. The second ophthalmic surgical instrument 44 includes mating structure 46 for locking engagement with lumen 30 and hub 36. In the embodiment shown in FIG. 5, mating structure 46 includes a flange 48 for threaded connection with hub 36. Again, lumen 30 is of sufficient length to frictionally fit on a surgical instrument, such as the first ophthalmic instrument 26 shown in FIG. 3, as well as for the locking engagement shown and described here at FIG. 5. FIG. 5 also shows a power cord 50 and an aspiration line 52.

It is also noted that structure 46 is of greater length than prior art mating structures for locking luers to accommodate lumen 30's extra length. Luer 25 may reliably be used on a prior art handpiece with a locking connection; however, the connection to the prior art handpiece will require reliance on a frictional fit connection, because lumen 30's length will not allow locking engagement with a prior art locking handpiece.

In this way, it has been shown that by providing luer 25 with a lumen 30 of sufficient length to frictionally seat and seal with a first ophthalmic surgical instrument while also providing a locking hub for locking engagement with a second ophthalmic surgical instrument. Therefore, the inventive tubing 22 with a sufficient lumen 30 length of luer 25 of the present invention, ophthalmic surgery has been made more efficient and allows the surgeon to use his preferred handpieces.

We claim:

1. A length of tubing having a luer connected thereto, the luer comprising:
a lumen of a length sufficient to frictionally engage with and seat against a mating recess of a first ophthalmic surgical instrument;
a locking hub for locking engagement with a mating structure of a second ophthalmic surgical instrument; and
thereby allowing the length of tubing to be alternatively connected to the first and second ophthalmic surgical instruments and allowing the length of tubing to be used on multiple instruments during a single surgical procedure.

2. The invention of claim 2, wherein the lumen length is at least 0.47 inches in order to sealingly seat with the mating recess.

3. The invention of claim 1, wherein the locking hub includes at least one thread for engaging a flange of the second ophthalmic instrument.
